(19) United States
(12) Patent Application Publication
(10) Pub. No.: US 2015/0297388 A1
(43) Pub. Date: Oct. 22, 2015

(54) ARM SUPPORT

(71) Applicant: Eric S. Peterson, Seattle, WA (US)

(72) Inventor: Eric S. Peterson, Seattle, WA (US)

(21) Appl. No.: 14/616,449

(22) Filed: Feb. 6, 2015

Related U.S. Application Data
(60) Provisional application No. 61/937,192, filed on Feb. 7, 2014.

(51) Int. Cl. A61F 5/37

(52) U.S. Cl. CPC A61F 5/3738

(57) ABSTRACT

A device for supporting the arms of a person reading a book or operating an electronic device. The device includes cradle means to support one or both forearms and strap means to transfer the weight to the shoulder or other torso area. The support strapping may be interconnected across the front or back of the person.

### TECHNICAL FIELD

This invention relates to the posture of the human body, and more particularly to a device for supporting the forearms in a position of comfort causing no neck strain when using an electronic device or reading. The device includes means, using the body, to support the person's forearms so that the device is comfortably in front of the face without requiring the head to be lowered, causing neck strain. Straps support a cradle for the forearm(s) and extend over one or both shoulders as needed.

### BACKGROUND OF THE INVENTION

According to doctors, there are 20 people a day treated for a syndrome called Text-Neck. It is caused by the positions people put their body in while texting, gaming and reading on hand-held devices such as smart phones and tablets.

The problems that arise are seen in neck strains, breathing difficulty and arm/shoulder pain. Add these issues together with the number of youngsters that use these devices daily and during the development stages of their bodies, and you will see the start of an epidemic in the near future.

Some of the statistics:
½ of all babies use a smart phone or tablet before the age of 2.
5-8 year olds use a smart phone or tablet on average over 2 hours per day.
86% of kids use smart phones or tablets for gaming.
72% of adults text message while teenagers text five times more. Sending and/or receiving 50 to 100 texts per day.

Neck strain as well as breathing difficulties occur due to the chin being dropped onto the upper chest while looking down at the device, generally held low or on the lap. The neck is strained in the cervical area while the airway is reduced when the neck is placed in this position.

### SUMMARY OF THE INVENTION

The device is a positioning/sling-type device that will allow a person to comfortably maintain their hand or hands positioned in front of their face/eyes and line of sight by elevating the elbows and holding them in place against the upper torso. The elbows are held in place comfortably in a cloth or hard-shelled cup attached to a flexible band and when relaxed will use the weight of the user's arms to hold the device in place against the torso and at an angle that will keep the hands elevated.

The device will allow for the hands and forearms to be elevated comfortably in the field of vision/in front of the face/eyes, therefore reducing the potential problems caused by dropping the chin down to view the hand held device or tablet.

Improvements over existing technology: Nothing in the market is made in this manner to achieve this result. There are stands and hard material-based products such as easel-like stands to hold a tablet or phone, but there is nothing portable and easily storable that can be used anyplace without a hard surface to hold it up or any specific necessary knowledge to achieve this end.

Groups of people and/or businesses that would use this invention: This invention can be used in and by the widest group of individuals available to the market, anyone from the youngest of children using their parent's phones or tablets for video games while riding in a car, train or plane or just sitting at home to teenagers who have proven to be the leading text messengers of the populace. Anyone would benefit who just wants to be more comfortable while reading a book or using their hand-held device while taking their hour-long bus ride home after work.

### BRIEF DESCRIPTION OF THE DRAWINGS

FIG. 1 is an isometric illustration of a person reading with the inventive device.

FIG. 2 is a frontal illustration of a person reading with the inventive device.

FIG. 3 is an illustration of the inventive device.

FIG. 4 is an illustration of a second embodiment of the inventive device.

FIG. 5 is an isometric of another species of the inventive device, one side in use.

FIG. 6 is a frontal view of the inventive device in use.

FIG. 7 is a back view of the inventive device.

FIG. 8 is a view of a portion of the inventive device.

FIG. 9 is a view of the entire inventive device.

FIG. 10 is a view of another adaptation of the inventive device.

### BEST MODE FOR CARRYING OUT THE INVENTION

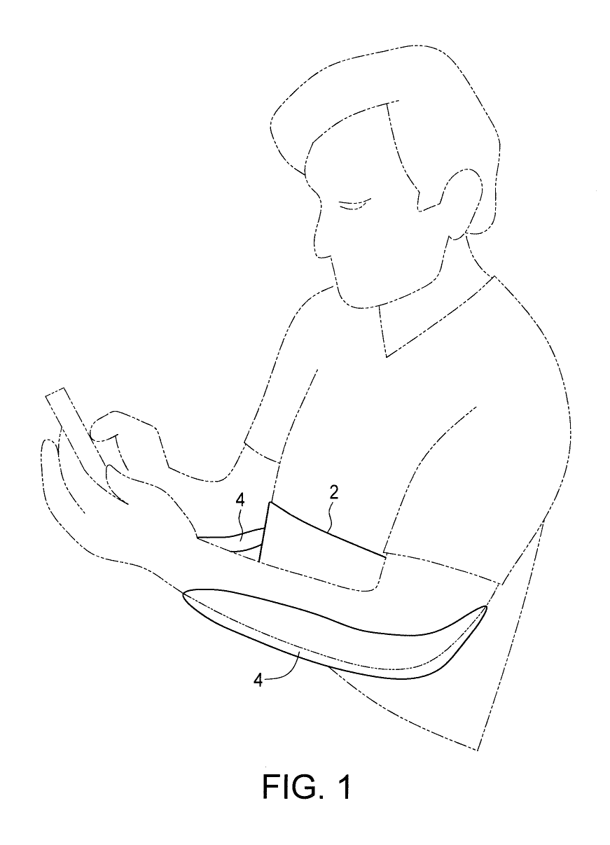

FIG. 1 illustrates the use of the inventive device which includes a base or panel 2 which rests against the wearer's abdomen. Secured to and extending outwardly from the base or panel 2 are a pair of extensions 4 which cup the person's elbows and support the forearms to place the phone or tablet in a position where the user's neck is comfortable and not cramped.

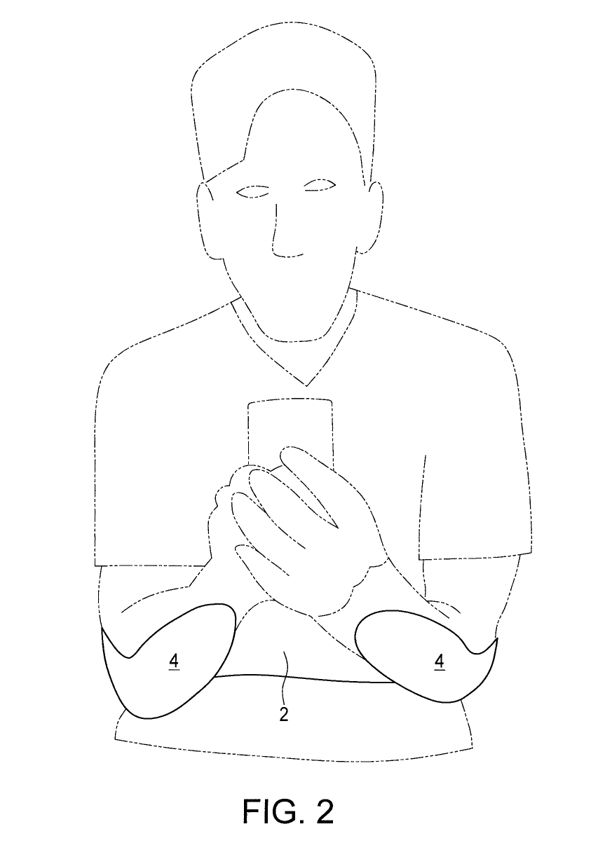

FIG. 2 is a face-on view illustrating how the invention improves posture and comfort.

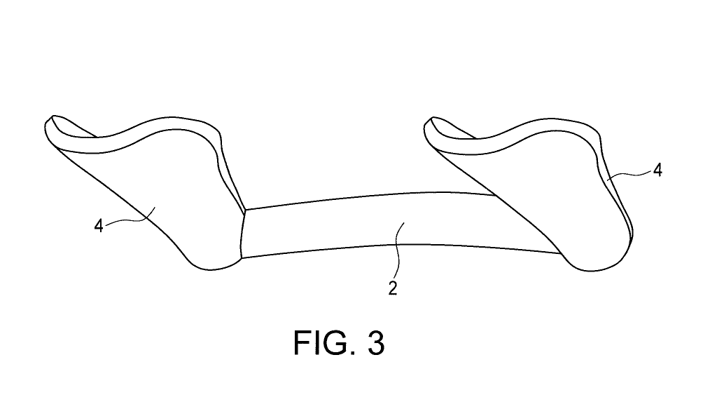

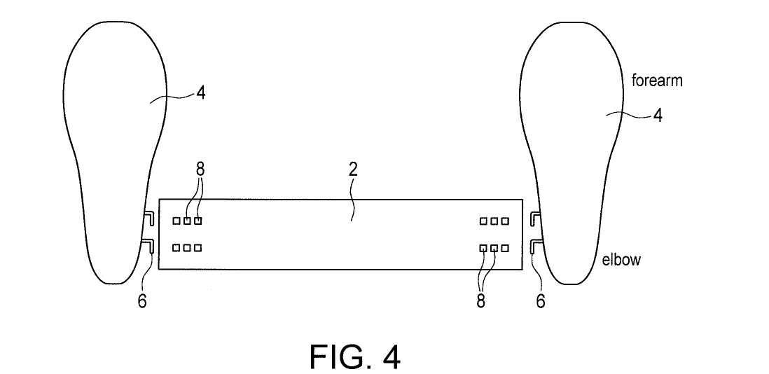

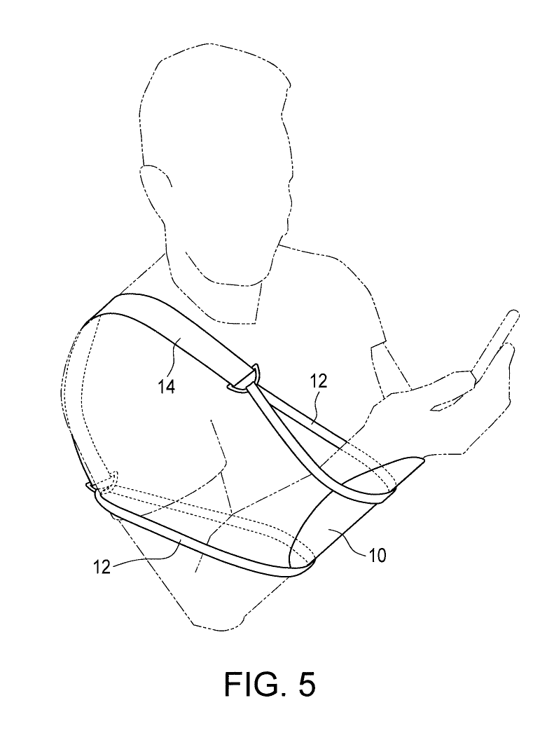

FIGS. 3 and 4 depict the inventive device by itself in two species, unitary in FIG. 4 and separable and adjustable in FIG. 5.

As seen in FIG. 4, the elbow and arm supports 4 are secured to the base 2 by means of hooks 6, which are inserted into openings 8.

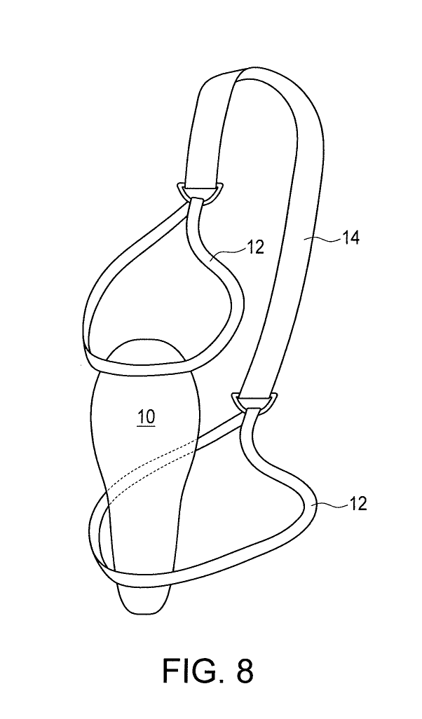

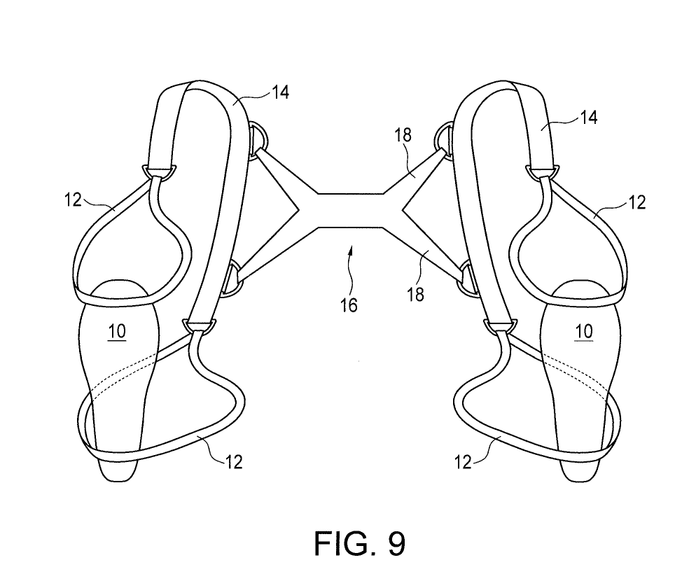

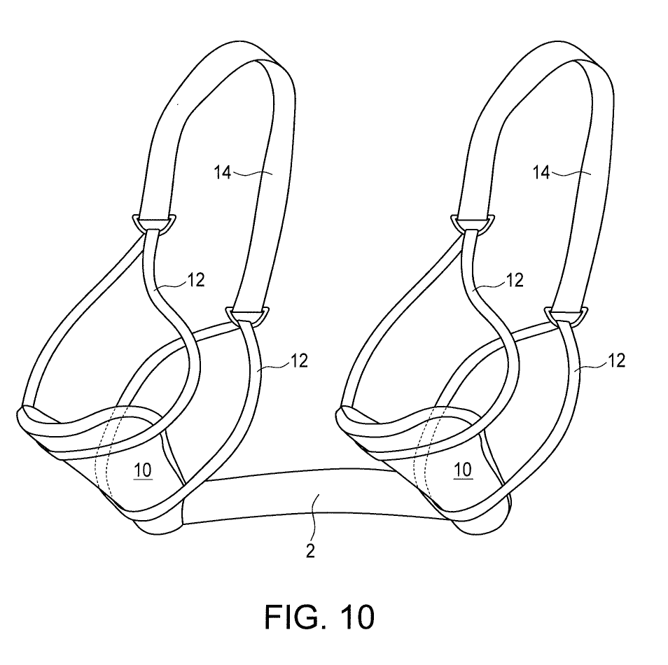

Reference is now had to FIGS. 5-10, wherein a second embodiment is shown.

As seen in FIG. 5, a person is shown using the device on one arm. The device consists of a pad 10 to support the forearm of the user supported by a pair of flexible loops 12 which are each supported by a fastener at each end of a flexible strap 14 designed to extend over the user's shoulder. It is to be understood that the straps 12, 14 are adjustable and may be elastic and the pad may be soft or a semi-rigid of plastic. Although the fastener means 16 are shown as simple loops, it is to be understood that they could equally well be adjustable to vary the length of either 12 or 14 or both.

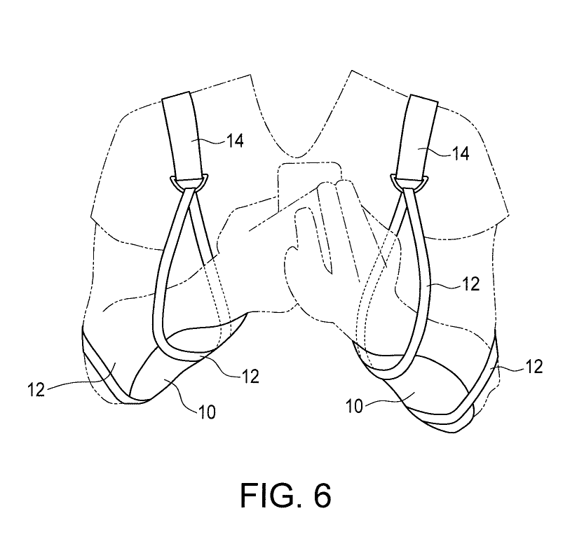

As seen in FIG. 6, a pair of the devices of FIG. 1 are used to support both hands shown holding a cell phone.

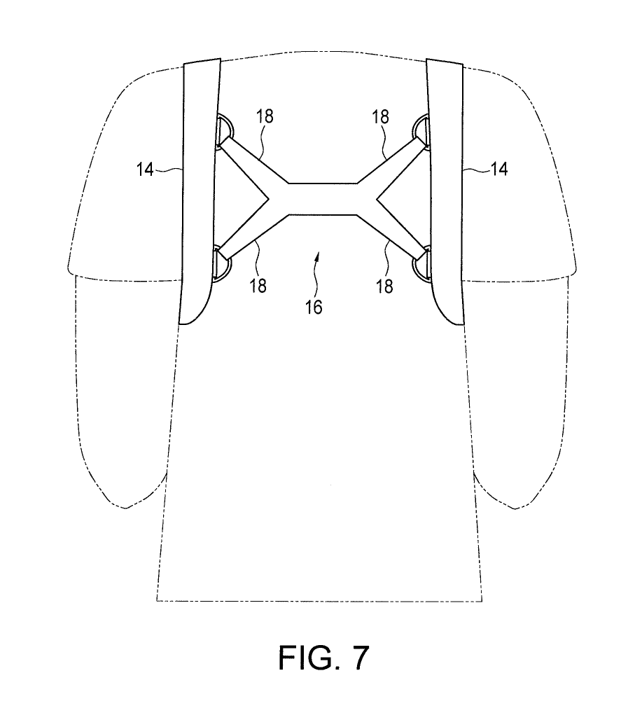

FIG. 7 depicts one possibility for stabilizing the device and incorporates a back device 16 including a central portion 18 and two pair of legs 18 secured to straps 14. Again, the interconnection could be adjustable and the straps in addition to being flexible could be elastic

It is to be understood that the securement and configuration are illustrative only, and it is contemplated that they may be modified.

Identified below are the parts/components of invention with descriptive name of each part:

Part 1—Base—Flat panel of neoprene that rests against the upper portion of the torso with downward pressure applied with placement of elbows into part 2 of invention.

Part 2—Elbow Cups—Attached to the outer edge of “The Base” where elbows fit to apply the downward pressure of The Base against the torso.

Part 3—Elbow Strap—Attached to “The Base” on both sides of the Elbow Cups—This is an adjustable strap used to determine the most comfortable angle of the elbows.

Part 4—Back Strap—Adjustable strap attached to the back side of The Base which will allow for the Invention to be used with and adjusted on the arms of seats to include but not limited to chairs, car booster seats and airline seats.

1—The Base—This is a flat piece of neoprene or like material that measures approximately 4-6 inches deep and 12-14 inches wide in a rectangular shape. The size will be determined when it is decided on whether to make it in one size fits all or various sizes for different age and body sizes.

2—The Elbow Cups are made from the same neoprene material as the base and are sewn on to the edges of the base to support the forearms and hands in using the invention. There may also be a version of the device that would use hard plastic cups for the elbows to allow for an attachment that will allow for the placement of a hand held device to be used with hands free ability.

3—Elbow Strap—A webbing strap, adjustable by pulling or loosening through a plastic attachment that will be attached to the base on both sides of the elbow cups which will allow for the user to adjust the angle of the elbows by tightening or loosening the webbing.

4—Back Strap—An adjustable webbing strap, adjustable by pulling or loosening through a plastic attachment that will be attached to the back of the base at both ends. The tightening or loosening of this strap will allow for the invention to be used/placed over the arms of seats to include but not limited to chairs, car booster seats or airplane seats.

The overall structure of the Invention: The structure of the invention is a one-piece neoprene or related type of material that is sewn together with no removable parts on the original version. There may also be a version of the Invention that allows for removable plastic elbow cups to be added by clipping them on to a grommet/hook which would be attached to the base. The webbing/straps are sewn on for making adjustments to the elbows and/or for the use in attaching the device to another structure such as the arms of a chair.

The relationship of the various parts: The parts working together are what make the invention viable, as the when the elbows placed in the elbow cups exert downward pressure against the torso, which allows the hands to be maintained at eye level or at a level that will allow for a better field of vision when using a hand-held device. The adjustment straps are used to allow the user better and more functionality depending on the situation that the device is being used in as well as the size of the person using it.

How the invention operates/functions: The invention functions by using the weight of a person's arms pressed against their upper torso to elevate the level of their hands while using a hand-held device or reading a book. The elbows are placed in a comfortable position and the aforementioned weight of the person's arms, holds it in place while seated or standing.

The unique features of the invention: They can be seen in the simplicity and natural body positions that will alleviate a potential problem with neck, arm and shoulder strain while doing something as common as reading a book, using a smartphone or other hand-held device.

Although a preferred embodiment of the invention has been disclosed for purposes of illustration, it should be understood that various changes, modifications and substitutions may be incorporated in the embodiment without departing from the spirit of the invention, which is defined by the claims which follow.

What is claimed is:

1. An arm support for retaining the human hand proximate the face, comprising:
a strap adapted to be placed extending over the shoulder;
a pair of connecting elements, one at each end of the strap;
a pair of flexible loops each secured to one of the connecting elements; and
a semi-rigid pad element for placement beneath the forearm supported by the flexible loops.
